(19) United States
(12) Patent Application Publication
(10) Pub. No.: US 2008/0256696 A1
(43) Pub. Date: Oct. 23, 2008

(54) Sample Collection Device

(76) Inventor: John Walsmley, Manitoba (CA)

Correspondence Address: ADE & COMPANY INC, PO BOX 28006 1795 HENDERSON HWY, WINNIPEG MANITOBA, R2G 4E9, CA

(21) Appl. No.: 10/598,667

(22) PCT Filed: Mar. 9, 2005
(86) PCT No.: PCT/CA2005/000358

§ 371 Date: Sep. 7, 2006

Related U.S. Application Data
(60) Provisional application No. 60/551,342, filed on Mar. 10, 2004.
(60) Provisional application No. 60/621,048, filed on Oct. 25, 2004.

(51) Int. Cl. E03D 9/00; A61B 10/00

(52) U.S. Cl. 4/420

(57) ABSTRACT

A novel device for the collection of stool and/or urine samples is arranged for use in a toilet having a rim and a bowl. The device has a sample collection platform arranged to receive the sample and an end wall extending from the sample collection platform at an angle to the sample collection platform, and the end wall includes attachment means for attaching the device to the bowl of the toilet.

### BACKGROUND OF THE INVENTION

Because stool and urine samples can provide valuable information regarding the health of a patient, medical practitioners often require samples for diagnostic purposes, for example, for detecting metabolite levels, microorganisms or occult blood. For these tests to be accurate, the sample must not be contaminated by toilet water.

Typically, most of the means known in the art for collecting these samples are inconvenient, embarrassing, complicated, awkward or distasteful. For example:

PCT Application WO 02/058561 teaches a device for collecting stool samples which comprises a collection dish and a support that hooks over the rim of the toilet bowl.

U.S. Pat. No. 5,463,782 teach a stool sample collection device which comprises a sheet of foldable material that is attached by adhesive to the toilet rim such that a cupping section extends down into the bowl.

U.S. Pat. No. 4,445,235 teaches a stool specimen collector that comprises a container suspended in the bowl that is attached to the upper surface of a toilet seat.

U.S. Pat. No. 4,309,782 teaches a stool specimen collector that comprises a bag-like structure that is beneath the back and side portions of a toilet seat.

U.S. Pat. No. 5,337,426 teaches a stool sample collector which comprises a sheet of flexible material which includes two slits therein which is then fitted over a toilet seat such that the portion of the sheet between the slits extends below the toilet seat.

U.S. Pat. No. 6,640,355 teaches a stool sample collector which comprises a collection structure that rests on the rim of the toilet bowl and has a telescoping arm that extends across the bowl and supports the front portion of the collection structure.

U.S. Pat. No. 6,653,149 teaches a method of collecting a stool sample wherein a hydrated polymer is added to the water in the toilet bowl prior to use such that the polymer forms a matrix which supports the sample above the water.

U.S. Pat. No. 6,434,762 teaches a stool collecting apparatus comprising a disposable strip which spans the toilet seat region and has a section with an opening for receiving the stool sample.

As can be seen, most of these devices must be placed either over or under the toilet seat, which may cause feelings of discomfort or strangeness for the user. Clearly, a device arranged for mounting beneath the seat but either on the rim or bowl of the toilet for collecting stool samples is needed.

### SUMMARY OF THE INVENTION

According to a first aspect of the invention, there is provided, in a toilet having a rim and a bowl, a stool sample collection device comprising:

a sample collection platform arranged to receive a stool sample from a user; and

an end wall extending from the sample collection platform at a right angle to the sample collection platform, said end wall including attachment means for attaching the device to the bowl of the toilet.

According to a second aspect of the invention, there is provided, in a toilet having a rim and a bowl, a urine and/or stool sample collection device comprising:

a receptacle having a cup arranged to receive urine and a spout extending upwardly from the cup at an acute angle;

a sample collection platform arranged to receive a stool sample from a user, said platform including an opening for inserting the cup therein; and

an end wall extending from the sample collection platform at a right angle to the sample collection platform, said end wall including attachment means for attaching the device to the bowl of the toilet.

### BRIEF DESCRIPTION OF THE DRAWINGS

FIG. 1 shows an embodiment of the device in breakaway in use within a toilet.

FIG. 2 shows the disassembled device.

FIG. 3 is a top view of the device.

FIG. 4 is a side view of the device.

FIG. 5 is a front view of the device.

FIG. 6 shows an alternative embodiment of the device in breakaway in use within a toilet.

FIG. 7 is a perspective view of the device.

FIG. 8 is a top view of the device.

FIG. 9 is a perspective view of the device.

FIG. 10 shows the shield of one embodiment of the device.

FIG. 11 shows the collector of one embodiment of the device.

FIG. 12 shows the base of one embodiment of the device.

FIG. 13 shows a top view of the partially assembled device arranged for urine collection.

FIG. 14 shows a top view of an alternative arrangement of the partially assembled device.

FIG. 15 is a top view of the base in flex position.

FIG. 16 is a side view of the base in flex position.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Referring to the drawings, the invention relates to a device 1 for urine sample collection from a female patient and/or stool sample collection. In another embodiment of the invention, the device 2 is arranged for stool sample collection.

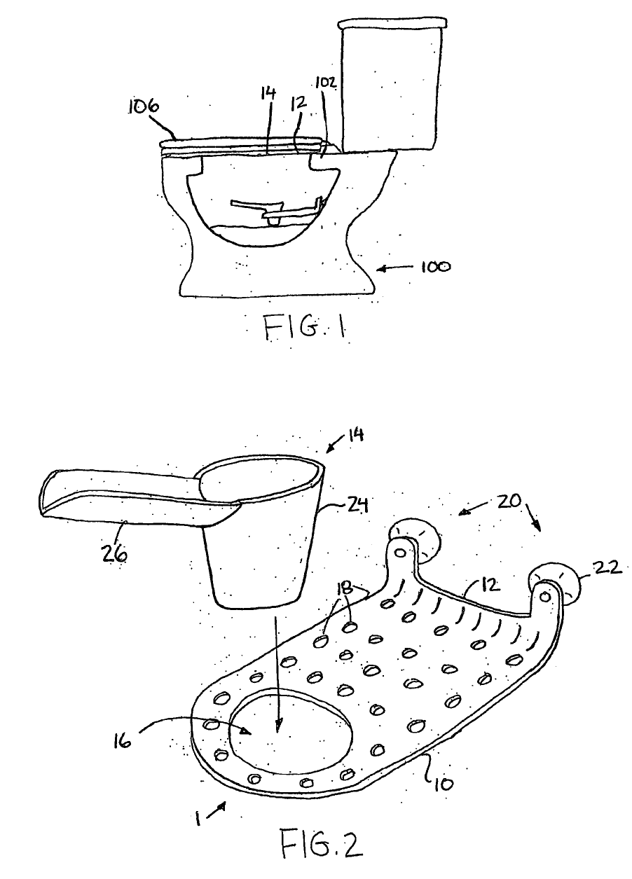

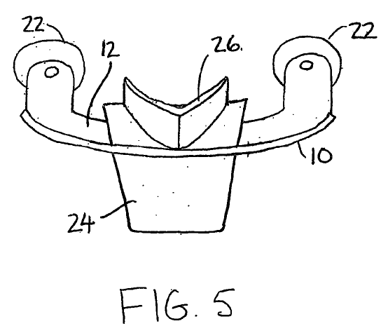

Referring to FIGS. 1-5, a device for urine sample collection from a female patient and/or stool sample collection 1 comprises a sample collection platform 10, an end wall 12 and a receptacle 14.

The collection platform 10 is arranged to collect the stool sample from the user and for insertion of the receptacle 14, as discussed below. Specifically, the collection platform 10 includes an opening 16 for receiving the receptacle 14.

In the embodiment shown in FIGS. 1-5, the collection platform 10 further includes a plurality of apertures 18 which extend through the platform. As will be appreciated by one of skill in the art, the apertures 18 provide for drainage of the sample deposited onto the collection platform 10. As can be seen in FIGS. 1-5, the apertures 18 can be evenly distributed throughout the collection platform 10 or in other embodiments may be localized to a specific region.

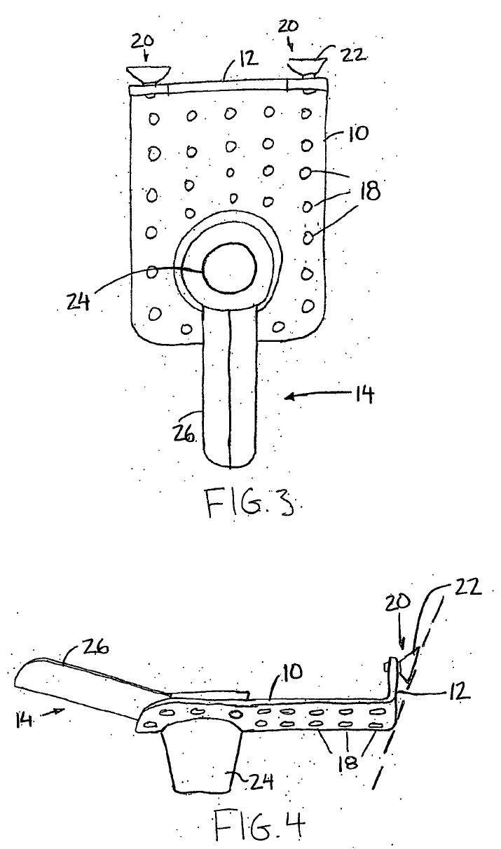

In the embodiments shown in FIGS. 2, 4 and 5, the collection platform 10 has a substantially concave shape which provides stiffness and integrity to the device 1 and also assists in retaining the stool sample on the collection platform 10. That is, as can be seen on referring to the diagrams, the middle of the platform is lower than either side of the platform and the platform is concave along the length thereof and across the width thereof. This arrangement also provides a three-point stabilized lever, as discussed below.

In the embodiments shown in FIGS. 1-5, the sidewall 12 extends upwardly from the collection platform 10 at an angle. In the embodiments shown in FIGS. 1-5, the sidewall 12 extends upwardly at a substantially right angle. The sidewall 12 includes attachment means 20 for attaching the device 1 to the side of the rim 102 or preferably bowl 104 of a toilet 100. In the embodiments shown in FIGS. 1-5, the attachment means 20 comprises two suction cups 22 laterally spaced and connected to the sidewall 12 at positions above the plane of the collection platform 10. As will be appreciated by one of skill in the art, the permissible angles of the end wall relative to the platform 10 are those that permit contact between the sidewall 12 and the bowl 104 when the attachment means 20 are engaged on the bowl such that the platform 10 is substantially horizontal within the bowl 104 so that the sample is retained thereon as discussed below. In a preferred embodiment, this angle is a substantially right angle, for example, an angle between 75°-105°, 80°-100°, 85°-95°, 86°-94°, 87°-93°, 88°-92° or 89°-91°. It is also of note that as discussed below, the device 1 or the device 2 is mounted to the bowl 104 of the toilet such that the collection platform 10 is above the level of the water in the toilet bowl.

In some embodiments, the receptacle 14 comprises a cup 24 and a spout 26. The spout 26 extends upwardly at an acute angle from the cup 24 and has a substantially convex shape for receiving urine thereon, thereby forming a channel or trough for collecting urine and directing the urine towards the cup 24. As can be seen from FIGS. 1-5, the spout 26 is arranged to direct fluid deposited onto the spout 26 downward and into the cup 24, as discussed below. In other embodiments, the receptacle 14 comprises a cup, for example, a disposable cup, for example, a paper cup such as those commonly used with drinking fountains, water coolers and the like. As will be appreciated by one of skill in the art, cups such as these are arranged to hold a suitable quantity of liquid and are also typically arranged to be disposable.

As discussed above, in the embodiments shown in FIGS. 1-5, the cup 24 is inserted into the opening 16 prior to use. In other embodiments, the cup 24 may be removably attached to the collection platform 10 such that the cup 24 can be easily separated from the collection platform 10. In other embodiments, the spout 26 may be connected to the cup 24 such that the spout 26 can be broken off and a lid or cap applied to the cup 24. Alternatively, in some embodiments, the cup 24 may not include a spout.

For use, the device 1 is removed from its packaging and the cup 24 is inserted into the opening 16. As will be appreciated by one of skill in the art, this is not necessary if the cup 24 was inserted into the opening 16 during packaging. The user may prepare the suction cups 22 for binding, for example, by applying water to the suction cups 22, for example, by dipping the suction cups 22 in the water in the toilet bowl 104. The user then positions the device 1 proximal to the side of the bowl 104 and applies pressure such that the suction cups 22 engage the bowl 104 at a position on the bowl 104 above the level of water in the toilet bowl 104. In this arrangement, the suction cups 22 adhere to the bowl 104 and the sidewall 12 contacts the bowl thereby forming a stabilizing lever. The female user then sits on the toilet seat 106, positions the spout 26 accordingly (if necessary) and urinates. During urination, the urine travels down the spout 26 and into the cup 24. The cup 24 can then be removed from the device 1. During defecation, the stool sample is deposited onto the collection platform 10 and held above the level of the water in the toilet bowl. The portion of the stool sample needed for analysis is then removed. It is of note that because the device 1 is mounted beneath the level of the toilet seat and is not mounted on and does not contact the upper surface of the rim and hence the underside of the toilet seat, the presence of the device is not noticeable to the user during use, that is, that unlike some prior art devices, the presence of the device 1 or 2 is not noticeable to the user while seated on the toilet seat.

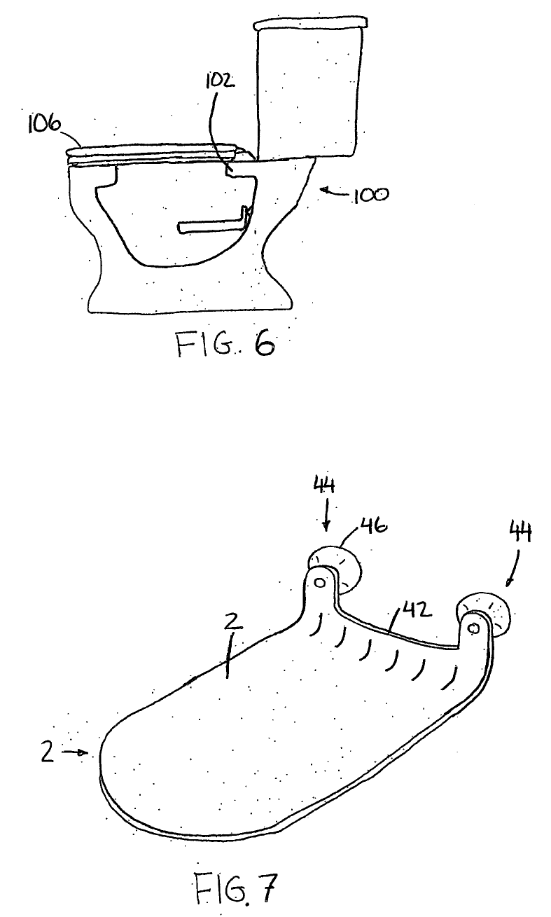

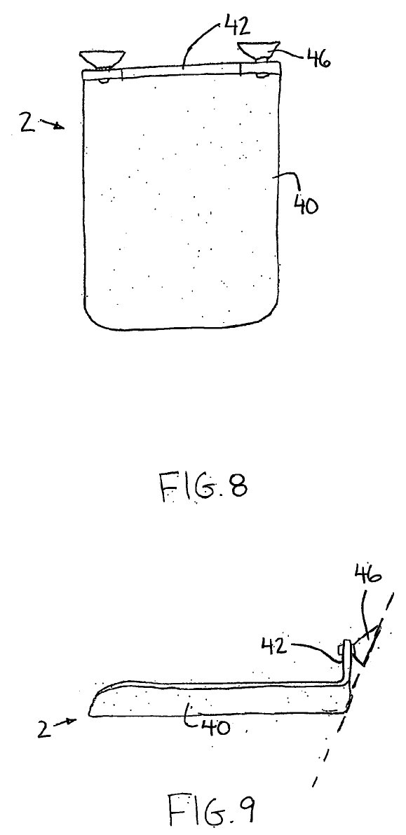

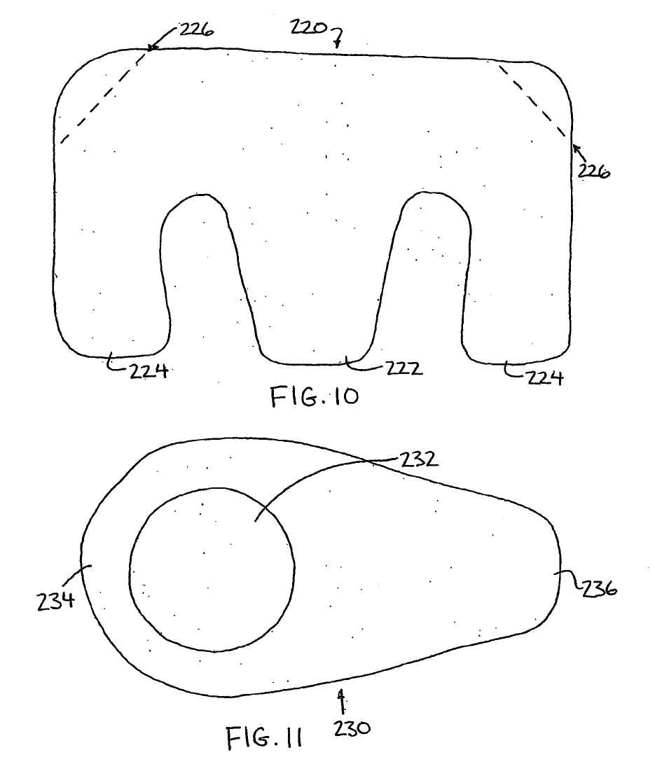

Referring to FIGS. 6-9, a device for stool sample collection 2 comprises a sample collection platform 40 and an end wall 42.

The collection platform 40 is arranged to collect the stool sample from the user, as discussed below and has a substantially concave shape which provides stiffness and integrity to the device 2 and also assists in retaining the stool sample on the collection platform 40, as discussed above.

In the embodiments shown in FIGS. 6-9, the sidewall 42 extends upwardly from the collection platform 40 at a substantially right angle, as discussed above. The sidewall 42 includes attachment means 44 for attaching the device 2 to the rim 102 or bowl 104 of a toilet 100. In the embodiments shown in FIGS. 6-9, the attachment means 44 comprises two suction cups 46 laterally spaced and connected to the sidewall 42 at positions above the plane of the collection platform 40.

The downward slope of the toilet bowl 104 picks up the lowest point on the curve of the platform 40 so that the platform 40 is stabilized to receive samples, as discussed above. Vertical points 42 are flexible plastic to work with the suction cups 46.

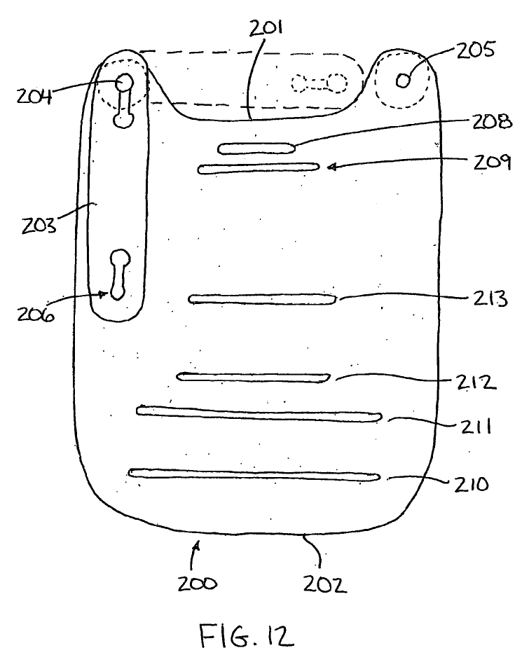

An alternative embodiment of the device is shown in FIGS. 10-16. Therein, the device comprises a base 200, a shield 220 and a collector 230. As can be seen in FIGS. 10-16, these components are arranged to be substantially flat for ease of packaging. In some embodiments, the components of the device are molded according to the desired shape in a flat configuration at a desired thickness, for example, 0.845 mm thick, thereby taking advantage of the material's ability to take a temporary shape that facilitates its functionality, as discussed below, as well as compactness for the purposes of packaging.

Referring to FIG. 12, the base 200 comprises a flexing end 201, an insertion end 202, a flap 203, male connector 205 and a plurality of slots.

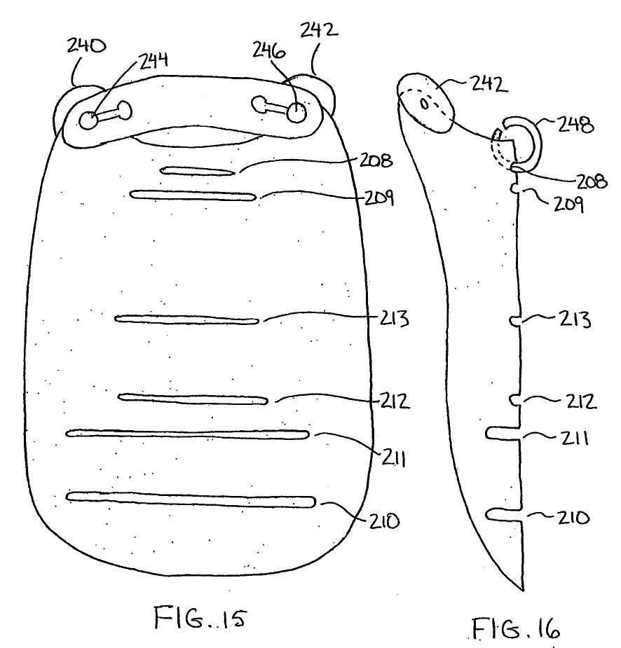

The flap 203 is mounted to the base 200 at a pivot point 204 such that the flap 203 pivots about the pivot point 204, as discussed below. The flap 203 further includes a female connector 206 which connects to the male connector 205. For assembly, the flap 203 is pivoted about the pivot point 204 and the flexible end 201 of the base is flexed or folded or bent so that the female connector 206 engages the male connector 205, as shown in FIGS. 15 and 16. As a result of this arrangement, the base 200 has a substantially concave shape for holding and retaining a sample thereon as discussed herein and the flap 203 extends across the flexing end 201.

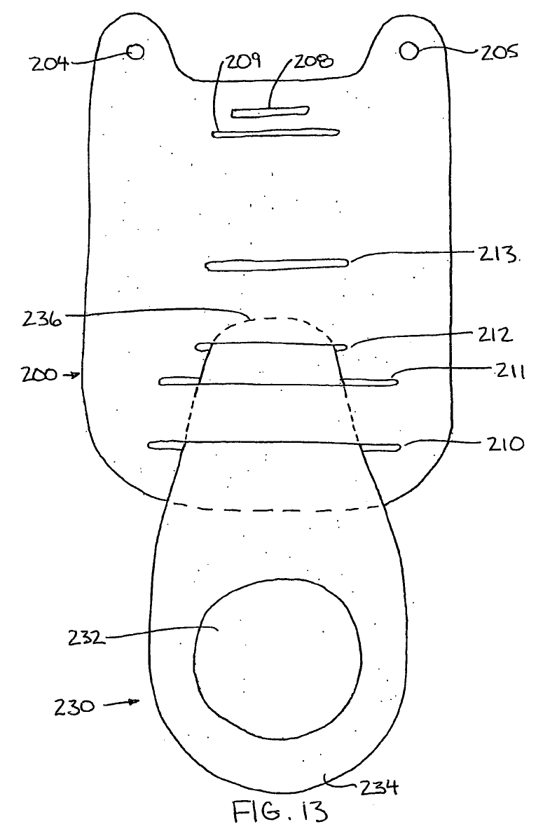

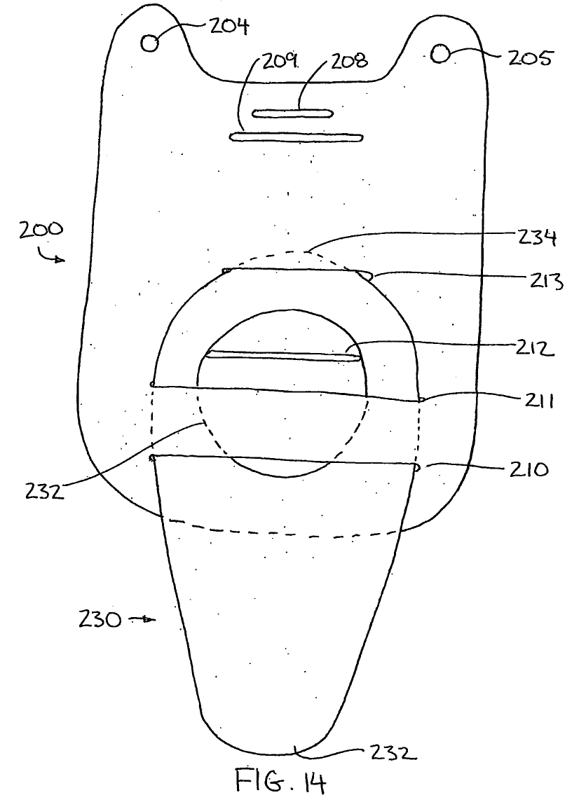

The plurality of slots include four insertion slots 210, 211, 212 and 213 for insertion of the collector 230 therein, as discussed below and as shown in FIGS. 13 and 14, ring slot 208 and shield slot 209. As can be seen in FIGS. 13 and 14, the slots extend laterally from side to side of the base 200 or platform.

The shield 220 includes a tongue 222 which is inserted into the shield slot 209 and flaps 224 which support the integrity of the device when the tongue 222 is inserted into the shield slot 209. Specifically, the shield 220 is a moisture shield which prevents toilet paper used to remove the stool from the device to the bowl from gathering water, rendering it useless. The shield 220 also includes folds 226, which can be folded downward to keep separation between the stool sample and the toilet bowl surface.

The collector 230 includes an opening 232, a first end 234 and a second end 236. As can be seen in FIG. 11, the opening 232 is closer to the first end 234 than the second end 236. As can be seen in FIGS. 13 and 14, either the first end 234 or the second end 236 of the collector 230 can be inserted through slots 210, 211, 212 and 213. In the embodiment shown in FIG. 14, the collector 230 adds strength and support to the device; in the embodiment shown in FIG. 13, the collector is arranged for urine collection, wherein a collection cup is inserted into the opening 232. As can be seen in FIGS. 13 and 14, the ends 234, 236 are inserted through the slots such that the collector 230 is effectively “weaved” through the slots, thereby stabilizing the desired confirmation. That is, the ends 234 or 236 may be inserted through the slots 210, 211, 212 and 213 such that the collector 230 is for example above the platform between slots 210 and 211 but beneath the platform between slots 211 and 212. As discussed above, the collection cup may be a paper cup, for example, a cup similar in shape and size to cups commonly supplied for water coolers, drinking fountains and the like. As will be appreciated by one of skill in the art, these cups are typically arranged to hold a volume of water that is similar to the volume of urine required for sample analysis. Furthermore, these cups are typically arranged to be disposable. It is of note that in some embodiments, a supply of suitable cups may be included with the device, for example, clipped together and inserted into the packaging for the device. Alternatively, the collection cup may include a spout as well as other features as discussed above.

In the embodiments shown in FIGS. 10-16, the device is assembled as follows. The flexing end 201 of the base 200 is manipulated as discussed above such that the female connector 206 on the flap 203 engages the male connector 205. As a result of this arrangement, the base 200 has a substantially concave shape for receiving the sample as discussed below. The shield 220 is then attached to the device by inserting the tongue 222 into the shield slot 209. As discussed above, the flaps 224 support the sides of the base 200, thereby stabilizing and strengthening the device. The folds 226 are folded downward, as discussed above. Next, one end of the collector 230 is inserted into the insertion slots 210, 211, 212 and 213, depending on the intended use. Specifically, as shown in FIG. 13, the second end 236 is inserted through the insertion slots 210, 211, 212 and 213 such that the opening 232 extends outwardly from the base 200 for urine sample collection. Alternatively, as shown in FIG. 14, the first end 234 is inserted through the insertion slots 210, 211, 212 and 213, thereby stabilizing the device. Next, suction cups 240, 242 are attached to the base 200. In a preferred embodiment, the suction cups 240, 242 are attached to the base via holes 244, 246 which are within the pivot point 204 and the male connector 205 respectively. Next, rings 248 are fitted onto the ring slot 208. As can be seen in FIG. 16, the rings 248 are in substantially the same plane as the base 200 and extend outwardly from the flex end. In use, the rings 248 rest against the bowl, thereby further stabilizing the device.

As will be appreciated by one of skill in the art, the above steps may be carried out in any sequence that results in a suitable device being assembled.

For use, the device 2 is removed from its packaging and if necessary is assembled as described above. The user may prepare the suction cups 46 or 240 and 242 for binding, for example, by applying water to the suction cups 46 or 240 and 242, for example, by dipping the suction cups 46 or 240 and 242 in the water in the toilet bowl 104. The user then positions the device 2 proximal to the bowl 104 and applies pressure such that the suction cups 46 or 240 and 242 engage the side of the bowl 104 at a position such that the base or platform is above the level of the water in the toilet bowl 104. It is also of note that the rings 248 will rest against the surface of the bowl 104, thereby providing further support and stabilization to the platform. The user then sits on the toilet seat 106 and defecates. The stool sample is deposited onto the collection platform 40 or base 200 and held above the level of the water in the toilet bowl. The portion of the stool sample needed for analysis is then removed. It is of note that the device 2 is arranged such that while using the device, male users may urinate without concerns about contaminating the sample or damaging the device as is the case with some of the prior art devices.

As will be appreciated by one of skill in the art, the device 1 for urine sample collection from a female patient and/or stool sample collection and the device 2 for stool sample collection may be made of any suitable material, for example, of a plastics material. It is of note that in some embodiments, the device 1 for urine sample collection from a female patient or the device 2 may be arranged as a disposable device for a single use. In yet other embodiments, the device 1 and the device 2 are arranged for multiple use.

While the preferred embodiments of the invention have been described above, it will be recognized and understood that various modifications may be made therein, and the appended claims are intended to cover all such modifications which may fall within the spirit and scope of the invention.

1. In a toilet having a rim and a bowl, a stool sample collection device comprising:
a sample collection platform arranged to receive a stool sample from a user; and
an end wall extending from the sample collection platform at an angle to the sample collection platform, said end wall including attachment means for attaching the device to the bowl of the toilet.

2. The device according to claim 1 wherein the sample collection platform has a substantially concave shape.

3. The device according to claim 1 wherein the attachment means comprises suction cups.

4. The device according to claim 3 wherein the device includes two suction cups spaced laterally apart on the end wall.

5. The device according to claim 1 wherein the platform includes a plurality of apertures.

6. The device according to claim 1 including at least one slot on the platform proximal to the end wall and at least one ring inserted through said slot.

7. In a toilet having a rim and a bowl, a urine and/or stool sample collection device comprising:
a receptacle having a cup arranged to receive urine;
a sample collection platform arranged to receive a stool sample from a user, said platform including an opening for inserting the cup therein; and
an end wall extending from the sample collection platform at a right angle to the sample collection platform, said end wall including attachment means for attaching the device to the bowl of the toilet.

8. The device according to claim 7 wherein the sample collection platform has a substantially concave shape.

9. The device according to claim 7 wherein the attachment means comprises suction cups.

10. The device according to claim 9 wherein the device includes two suction cups spaced laterally apart on the end wall.

11. The device according to claim 7 wherein the cup includes a spout extending upwardly from the cup at an acute angle
